(19) United States
(12) Patent Application Publication
(10) Pub. No.: US 2017/0027603 A1
(43) Pub. Date: Feb. 2, 2017

(54) FLEXIBLE DEVICES FOR BLUNT DISSECTION AND RELATED METHODS

(71) Applicant: AMS RESEARCH CORPORATION, Minnetonka, MN (US)

(72) Inventor: Prabhaka Pandey, Glendale, AZ (US)

(21) Appl. No.: 15/302,749

(22) PCT Filed: Apr. 7, 2015
(86) PCT No.: PCT/US15/24686

§ 371 Date: Oct. 7, 2016

Related U.S. Application Data
(60) Provisional application No. 61/976,769, filed on Apr. 8, 2014.

(51) Int. Cl. A61B 17/32; A61B 90/00; A61B 17/34

(52) U.S. Cl. CPC A61B 17/320016; A61B 17/3421; A61B 90/361; A61B 2217/005; A61B 2217/007; A61B 2017/320048; A61B 2017/00805

(57) ABSTRACT

Described are devices and methods for forming a surgical space within a body of a patient by blunt dissection, including devices that include a blunt dissection device and optionally one or more of an insufflation lumen, access lumen, and viewing device.

### PRIORITY CLAM TO PROVISIONAL APPLICATION

This application claims priority to U.S. Provisional Patent Application No. 61/976,769, filed Apr. 8, 2014, the entire contents of which are incorporated herein by reference in its entirety.

### TECHNICAL FIELD

The following description relates to devices and methods for forming a surgical space within a body of a patient, in particular, devices that include a blunt dissection mechanism and optionally one or more of an insufflation lumen, access lumen, and a device for viewing a surgical site, as well as methods of using such devices for forming a surgical space within a body of a patient.

### BACKGROUND

During many or most surgical procedures it is necessary to manipulate tissue of a patient to form a surgical space within a patient, for accessing a surgical site. One technique for moving or separating tissue in certain types of surgical procedures, to create space at a surgical site, can be referred to as “dissection,” which includes dissection by cutting with a sharp instrument as well as “blunt dissection” by use of a blunt surgical instrument or a finger of a surgeon. Blunt dissection can be useful for dissection techniques used to separate and create space between distinct tissues (e.g., muscle and fat, muscle and fascia, muscle and bone, tissue of a peritoneum and adjacent tissue), between two adjacent organs such as a urethra and a vagina, etc. Common advantages of blunt dissection compared to the use of a cutting instrument can be reduced levels of trauma and bleeding. Some procedures that involve blunt dissection are laparoscopic or endoscopic. These procedures may also include a step of insufflation of a surgical site, as well as optical viewing of the surgical site.

### SUMMARY

The surgical arts involve an ongoing pursuit for new and better devices and methods for performing surgical procedures, especially with respect to improved results (efficacy), patient safety, and reduced costs. Described herein are devices and methods that involve improvements in blunt dissection of tissue techniques.

The described blunt dissection devices can include a flexible shaft that allows a device to be useful in either a laparoscopic or a non-laparoscopic surgical procedure such as a transvaginal procedure. Preferred embodiments of devices can also include: an insufflation lumen to allow insufflation of tissue surrounding a surgical site after a step of blunt dissection and during a surgical procedure that uses the device; an optical viewing feature that can allow for viewing of the surgical site during a procedure, such as with the surgical site being dissected by a blunt dissection step and then expanded by insufflation; and an access lumen extending along a length of the device shaft, allowing a surgeon to pass surgical items or tools to the dissected and optionally expanded (insufflated) surgical site during the surgical procedure. A preferred device that includes a combination of a blunt dissection feature, an insufflation lumen, and an optical viewing feature (viewing device), can allow for open access of an expanded (insufflated) surgical space, full viewing of the surgical site and steps of the procedure, and reduced trauma and bleeding by use of blunt dissection as opposed to dissection by cutting tissue.

According to certain embodiments of the novel devices, a surgeon is able to: dissect a surgical site with advantageous blunt dissection techniques; insufflate the site to create an expanded surgical space; view the surgical space and a surgical site within the insufflated surgical space; and optionally pass surgical items such as an implant item, surgical tool, endoscope, or catheter insert along an access lumen of the device, during a surgical procedure, to the surgical site, without removing the blunt dissection (and insufflation) device.

Advantageously, the ability to dissect a surgical site, expand the surgical space by insufflation, and then view the surgical space and perform a surgical procedure with full vision of the surgical space, will result in completely new techniques and advantages for performing a range of surgical procedures, particularly in surgical procedures that are presently performed without full vision of an insufflated surgical space.

Many surgical procedures, including procedures performed in a pelvic space, are performed without insufflation or without vision of the surgical space. For example, transvaginal procedures for placing an implant in a female patient to treat a condition of incontinence, prolapse, avulsion, etc., are conventionally performed without insufflation and without viewing the surgical site using an optical device or tool. The present devices and methods, embodiments of which now allow for full transvaginal viewing and insufflation of a pelvic surgery site (among other anatomical locations), will now open up entirely new and not previously contemplated techniques, treatments, and advantages, when used in transvaginal surgical techniques in the pelvic region, as well as other non-transvaginal techniques at locations other than a pelvic region. Potential advantages will improve the safety of any such procedure, as well as efficacy. With full viewing of the surgical site a surgeon will have the utmost ability to avoid accidental contact with nerves, blood vessels, or sensitive tissue, thus avoiding unnecessary damage or trauma. Full viewing also allows precise placement of sutures, tissue anchors, and implant support structures, to improve the success rate of surgeries.

Devices for blunt dissection of tissue, such as expandable balloon blunt dissection devices, are known to be useful in certain surgical procedures such as extraperitoneal (e.g., abdominal) laparoscopic procedures. Blunt dissection, e.g., using a three-dimensionally expandable surface such as an expandable balloon to separate tissue or to separate adjacent layers of tissue, is not commonly used or understood to be useful in a variety of other types of surgical procedures including transvaginal procedures for placing an implant in a pelvic region of a female patient for treating a condition of incontinence, prolapse, levator avulsion. The present inventor has performed balloon dissection using a Foley balloon catheter in transvaginal procedures of treating conditions of an anterior pelvic region in a female patient, as well as in retropubic dissection to place a reservoir of a penile implant or an artificial urinary sphincter. But a Foley catheter is of course not designed for the purpose of blunt dissection. The description that follows includes examples of new and advantageous devices that have features that are specifically designed to be used for blunt dissection of tissue of a pelvic region of a male or female patient. Exemplary devices are especially effective for blunt dissection of tissue of a female pelvic region, e.g., an anterior pelvic region in a transvaginal procedure. Certain examples of conditions that can be treated include female stress urinary incontinence, pelvic organ prolapse such as cystocele, rectocele, enterocele, vaginal vault prolapse, and defects such as levator avulsion.

However, while the following often describes examples of devices and methods in the context of treating pelvic conditions of a female patient by a transvaginal method, the devices are understood to be useful for blunt dissection of tissue at other locations of a male or female patient anatomy. The described devices and methods can be useful at any location of tissue that can be treated by blunt dissection, including locations at which blunt dissection has been useful in past procedures and practices as well as other locations at which blunt dissection will now be understood to be either useful or advantageous when performed using a device as described herein. These devices and methods can generally be useful for performing blunt dissection between a tissue or anatomical structure in a region of a surgical site at which a surgical procedure desirably takes place, to produce an opening or surgical space adjacent to the tissue or anatomical structure (i.e., adjacent to a surgical site). The surgical space produced by the blunt dissection can be useful during the surgical procedure to allow placement of a surgical instrument; for locating or staging a surgical implant before or during attachment to tissue at the surgical site; to allow viewing of the surgical site, implant, tissue, or anatomical structure; or for any other purpose that will allow the surgeon to perform a desired surgical step at the surgical site.

The device generally includes a flexible shaft, a blunt dissection device (e.g., inflatable balloon) at a distal end, an optional insufflation lumen, an optional access lumen, and an optional optical viewing device that may be either integral to the flexible shaft or insertable into and separable from an access lumen of the flexible shaft. The device can be used or provided in combination with one or more of an endoscope that includes a shaft, and that is adapted to allow the distal end of the endoscope shaft to pass through an access lumen of the device to the distal end of the device. Alternately or in addition, the device can be used or provided in combination with one or more catheter inserts that include a shaft, and that are each adapted to allow the distal end of the catheter insert shaft to pass through an access lumen of the device and become located at the distal end of the device. The catheter insert can be one that allows for placement of suction at the surgical site, delivery of an irrigation fluid at the surgical site, or that allows for viewing of the surgical site.

As indicated, methods of using the described devices include transvaginal methods for treating a female pelvic condition. In these methods the device allows for blunt dissection at a location referred to herein as a “submucosal plane” or “interfacial plane.” The tissue at this dissection site is relatively avascular. Advantageously, blunt dissection can be performed at this region with a greatly reduce amount of bleeding and blood loss as compared to techniques of dissecting the same pelvic region tissue by cutting. The total amount of patient blood loss is greatly decreased using blunt dissection. An added advantage is that the time required to perform a surgical procedure on the patient, such as a procedure to place a supportive implant, is reduced substantially. As a single comparison, a transvaginal method for placing an anterior vaginal sling using blunt dissection at the interfacial plane, as described, may take an amount of time in a range of about forty minutes, compared to about ninety minutes for previous techniques using a sharp cutting device for dissection. And the amount of blood loss is dramatically reduced by the blunt dissection method.

The methods also include non-transvaginal methods, especially extraperitoneal methods for treating a male or a female patient. An example of a non-transvaginal application is an extraperitoneal anterior abdominal method for accessing and treating abdominal muscles or adjacent tissue, such as for treating a hernia condition. Another example is an extraperitoneal posterior pelvic region method for accessing and treating a muscles, tissue, or organ of the posterior pelvic region of a male or female patient, e.g., rectum, sphincter, or adjacent muscle or tissue. Still another example is an extraperitoneal kidney access method for accessing and treating a kidney or adjacent tissue. Yet another example can be an extraperitoneal method for developing a retropubic space for reservoir placement of a three-piece implantable penile prosthesis or artificial urinary sphincter in a male patient. Advantageously, for use in treating a variety of pelvic condition, the described devices and methods are adaptable to procedures that do not require puncturing the parietal peritoneum that surrounds the pelvic region.

In one aspect, the invention relates to a blunt dissection device that includes a proximal device end, a distal device end, a flexible shaft extending along a length between the proximal device end and the distal device end, an expandable surface extending along a length of the shaft at the distal device end, expandable from the shaft, a lumen extending along a length of the shaft in operative communication with the expandable surface, and an insufflation lumen extending along a length of the shaft between the proximal end and the distal end.

In another aspect the invention relates to a blunt dissection device that includes a proximal device end, a distal device end, a flexible shaft extending along a length between the proximal device end and the distal device end, an expandable surface extending along a length of the shaft at the distal device end, expandable from the shaft, a lumen extending along a length of the shaft in operative communication with the expandable surface, and an access lumen within the shaft extending from the proximal device end to an access opening at a distal end of the shaft.

In another aspect, the invention relates to a method of blunt dissection. The method includes: providing a blunt dissection device as described, making an incision in a patient in a patient, passing the device distal end through the incision, advancing the device distal end to place the expandable surface at a dissection site, and expanding the expandable surface to cause blunt dissection of tissue at the dissection site.

### BRIEF DESCRIPTION OF THE DRAWINGS

FIGS. 1A and 1B show examples of a device as described, and catheter inserts as described.

FIGS. 2A, 2B, and 2C show pelvic anatomy and an exemplary blunt dissection site of a female patient.

FIG. 3 shows pelvic anatomy and an exemplary blunt dissection site of a female patient.

FIG. 4 shows pelvic anatomy and an exemplary blunt dissection site of a male patient.

FIG. 5 shows pelvic anatomy and an exemplary posterior blunt dissection site.

FIG. 6 shows pelvic anatomy and an exemplary blunt dissection site at a region of a kidney.

### DETAILED DESCRIPTION

The following describes devices, systems, and methods that involve blunt dissection devices and uses of a blunt dissection device for surgical procedures that involve blunt dissection in addition to other surgical steps.

The devices and methods, in various embodiments and procedures, can be useful for treating pelvic conditions by methods that involve blunt dissection of pelvic floor tissue. Exemplary devices can provide surgical access to tissue of a pelvic region in a male or a female patient for treatment of a pelvic condition by a technique that may involve placement of an implant such as a sling, artificial sphincter, or other implantable device (such as a component of an implantable penile prosthesis). The procedure may treat or operate on tissue of an anterior pelvic region, a posterior pelvic region, or surrounding or related tissue (e.g., tissue of a levator muscle, urethra, anal or urethral sphincter). Specific examples of pelvic conditions that may be treated include incontinence (various forms such as fecal incontinence, stress urinary incontinence, urge incontinence, mixed incontinence, etc., in a male or female patient), vaginal prolapse (including various forms of anterior, posterior, and apical prolapse referred to as enterocele, cystocele, rectocele, apical or vault prolapse, uterine descent, etc.); conditions of the pelvic floor and result from weakness or trauma of pelvic floor muscles such as the levator (“levator ani”) or coccygeus muscle (collectively the pelvic floor), and other conditions caused by muscle and ligament weakness. The methods useful to treat a female patient can be performed via a transvaginal incision, optionally using a single incision in the vaginal tissue with no external incision. For male anatomy, methods of treating urinary incontinence can involve a single external perineal incision. But, while the described devices and methods are useful and described in the context of treating male and female pelvic conditions, the methods and devices are not limited to uses that relate to pelvic conditions. The described devices and method can also be useful for surgical treatments of non-pelvic tissues and non-pelvic conditions.

In addition to blunt dissection, certain embodiments of the devices are capable of performing other surgical steps such as insufflation of a surgical space (e.g., after blunt dissection), direct viewing of a surgical space using an optical viewing feature of the blunt dissection device, and accessing a surgical site through an access lumen in the shaft of the blunt dissection through which surgical items may be passed and delivered to the surgical site.

The described blunt dissection devices and methods involve the use of an expandable surface for blunt dissection. The expandable surface may be an inflatable balloon or other expanding surface such as an expandable and retractable metal cage. The blunt dissection device can be used in a manner that produces reduced or minimal tissue trauma relative to dissection by cutting tissue using a sharpened instrument such as a blade, knife, scalpel, etc. In relation to alternate blunt dissection techniques, e.g., digital techniques that involve only the use of a surgeon's fingers, the described use of an expandable surface to effect blunt dissection can be more convenient, efficient, and less physically demanding for a surgeon who may be required to perform multiple such blunt dissections in a day or over a short period. Without a device as described, a surgeon would be required to perform the blunt dissections digitally, which may result in an undesired level of physical strain and exertion for the surgeon if performed multiple times in a single day.

Most surgical procedures require dissection and displacement of some tissue at or adjacent to a surgical site, to create a surgical space and access to relevant tissue and anatomy of a surgical site. Blunt dissection can be useful to assist in preparing a surgical space at a surgical site that will be the subject of a tissue repair or that will be used for placing an implant to support or reinforcing damaged tissue, including but not limited to tissue of a pelvic region (e.g., vaginal tissue, a urethra or bladder or bladder neck, pelvic floor muscle, a urinary or anal sphincter, tissue, etc.).

For a male or a female patient, treatment of a pelvic condition may include treatment of urinary or fecal incontinence, vaginal prolapse (in a female patient, e.g., anterior, posterior, vaginal vault prolapse, enterocele, cystocele, rectocele, vaginal vault prolapse), levator avulsion, or combinations of these. A procedure may involve placing a supportive implant at the treated tissue and extending a portion of the implant to supportive tissue in the pelvic region at which the extended portion of the implant is attached to provide support. A procedure may require access to tissues relevant to the conditions, and adjacent tissue to which the implant can be attached for support. Examples of relevant tissues of a pelvic region include tissues of a pubic bone, ischial spine, sacrospinous ligament, white line (arcus tendineus), pelvic surface of the obturator foramen covered with obturator fascia, bladder or bladder neck, urethra and adjacent fascia, levator muscle, perineal body, uterosacral ligament, sacrum, or coccyx bone, among others.

In certain embodiments of methods as described, including transvaginal methods of treating a pelvic condition in a female patient, a blunt dissection step can include blunt dissection of a space within of the pelvic region referred to herein as the “interfacial plane” or the “submucosal plane.” This region of tissue in a female patient can be considered to be approximately a planar region that includes the location between anterior vaginal tissue and the bladder and bladder neck (at a medial portion of the plane), and locations extending on both sides of the patient in an anterior direction to the pubic bone, laterally to the opposed obturator foramen, arcus tendineus and ischial spine, and posteriorly to the sacrospinous ligament and sacral region, including the coccyx bone. This planar region includes an interface between non-muscle tissues (generally fascia or fatty tissues), is generally avascular and, advantageously, can be separated by blunt dissection without the need for cutting tissue using a sharp cutting instrument.

Referring now to the described blunt dissection devices, they include generally a proximal end, a distal end, and a shaft extending between the proximal and the distal ends. At the distal end is an expandable surface that can be alternately expanded (increased in volume) and retracted (decreased in volume) in a manner that is effective for blunt dissection of tissue. The expandable surface can be expanded to create space between tissues, advanced to press against tissue to separate and move the tissue to create space between tissues, or both, optionally also being retracted between steps of expanding and pressing against tissue.

Preferably, the shaft is non-rigid, non-metal, polymeric and flexible. The term “flexible” in reference to the shaft refers to a shaft that is sufficiently pliable to allow bending and flexing of the shaft such that the shaft (at least a distal end thereof) is capable of being inserted through an incision in a patient (e.g., an external skin incision or a vaginal incision), advanced through the incision to tissue of a surgical site, and then guided to the surgical site, with the flexible shaft being sufficiently soft and pliable to conform or partially conform to a patient's anatomy during insertion and use during a surgical procedure, as would a standard flexible polymeric shaft of a variety of known catheter devices (e.g., a urinary catheter).

The shaft can have dimensions and mechanical properties (degree of flexibility) based on the utility for which the shaft is designed, e.g., the type of procedure for which the device will be used. Generally the shaft will exhibit a relatively uniform outer diameter along the length between the proximal and distal ends, comparable to 20 FR (French Unit) silicone, rubber, or polyurethane shaft of a Foley catheter. Examples of useful shaft outer diameters may be, e.g., from about 4 to 10 millimeters in diameter, such as from 5 to 8 millimeters in diameter.

The expandable surface can be a surface that can be expanded to increase in size in three dimensions, i.e., increase in volume, and alternately retracted to decrease in size to an original volume. Examples include inflatable balloon surfaces as well as mechanically extendable and retractable components such as expandable metal cages, and the like, any of which can be extended from a shaft of a blunt dissection device to place pressure on internal tissue and move the tissue to create space between otherwise adjacent or connected tissues. The expandable surface (e.g., balloon) may be round, e.g., having a circular, oval, or spherical shape in two or three dimensions, or may alternately exhibit a round (e.g.,) circular flat disc (“discoid”) shape. The volume of the expandable surface in an expanded state may be any volume desired or useful to perform a blunt dissection step at a particular dissection site; an example of a volume of an expandable surface in an expanded state for use in a transvaginal procedure can be in a range from 50 to 100 cubic centimeters, e.g., from about 60 to about 90 cubic centimeters. Desirably, to facilitate blunt dissection using the expanded surface, including by advancing the expanded surface distally to separate tissue, the a location along the shaft at which a distal end of the expandable surface meets the shaft can be near the shaft terminus, e.g., a distal end of the expandable surface can be located a distance of not more than 5 millimeters from the terminus.

Extending along the shaft between the proximal end and the expandable surface at the distal end is a lumen that includes a continuous channel, a mechanism such as a wire, or other means for actuating the expandable surface to cause the expandable surface to alternately expand and retract as desired. For an expandable surface in the form of an inflatable balloon, the lumen can be an inflation lumen in the form of an open or openable channel through which an inflation fluid (e.g., air, another gas, or a liquid) can be passed under pressure from the proximal end to the distal end to cause the balloon to expand and increase in size.

An optional feature of the blunt dissection device can be an additional lumen, referred to as an insufflation lumen, that extends between the proximal end and the distal end, to an opening at the distal end. The insufflation lumen can be a continuous open channel, optionally including a one-way check valve that is sufficient to prevent insufflation gas from escaping an insufflated surgical site. The insufflation lumen can be adapted and used to supply pressurized insufflation gas to an opening (insufflation opening) at the distal end of the device for delivery of the insufflation gas to a surgical site, e.g., during or after blunt dissection. The insufflation gas can preferably be carbon dioxide at a pressure sufficient to insufflate a surgical space, e.g., an internal insufflation pressure in a range up to about 20 mm Hg, e.g., from 10 to 20 mm Hg. The proximal end of the device can include or can be connected to a source of the pressurized insufflation gas.

Another optional feature of the blunt dissection device can be still one more additional lumen, referred to as an access lumen, that extends along the length of the shaft of the device between the proximal end and the distal end, to another opening on the distal end, preferably an access lumen opening that opens at the terminus of the shaft. Desirably, the opening can be oriented transversely to a longitudinal axis of the shaft, at the terminus, i.e., as opposed to a lateral opening that would pass through a sidewall of the shaft. The access lumen can be a continuously open channel, optionally including a one-way check valve to prevent insufflation gas from escaping an insufflated surgical site. The access lumen can preferably run along an internal location of the shaft and be sized to allow a medical instrument or a surgical item to be passed through the access lumen to be presented to a surgical site by being passed through the access lumen along the length of the shaft and then exiting the shaft through the access lumen opening at the shaft terminus. In embodiments of devices and methods that involve insufflation of a surgical space, the access lumen can preferably include a check valve or other type of closure that will withstand an insufflation pressure, and that will allow an item such as a catheter insert or an endoscope to be passed through the access lumen during a surgical procedure while maintaining an insufflation pressure at the surgical space.

An access lumen can preferably exhibit a relatively uniform diameter along the length between the proximal end and the distal end of the shaft, with examples of a useful diameter of an access lumen being, e.g., at least 3, 4, or 5 millimeters, such as from about 3 to about 8, e.g., from about 5 to 8 millimeters in diameter. Desirably the access lumen can be sized to allow drainage of fluid (e.g., blood) and to allow a 15 Fr endoscope to pass through the access lumen to become located outside of a distal access lumen opening at a distal end of the blunt dissection device, during a surgical procedure, to allow the endoscope to be used to visualize a surgical site or surgical space, or to allow another type of surgical device or tool such as a catheter insert (having a suction, viewing device, or irrigation functionality) to be passed through the access lumen to reach the surgical site.

In certain embodiments, a blunt dissection device may include a single access lumen through which one or multiple surgical items (e.g., catheter inserts) are passed, e.g., one at a time, sequentially or alternately during a surgical procedure. In other embodiments a blunt dissection device may include two or more parallel but separated access lumens, each separate access lumen extending the length of the shaft from the proximal end to the distal end. A first access lumen may be used to pass a first surgical item such as a viewing device. The second access lumen may be used for another surgical item, i.e., for passing a surgical item that provides provide suction, irrigation, a surgical scissors, an implant or implant piece to a surgical site such as a sacrospinous ligament.

Also optionally the blunt dissection device may include a steering feature that allows the distal end of the shaft or a catheter insert passed through the shaft, to be steered. As another option, an access lumen may be telescoping or extendable beyond the terminus of the shaft, and steerable when it is extended beyond the terminus.

The blunt dissection device can include an integrated or a separate viewing device that can be useful to view of surgical site at the distal end of the blunt dissection device. The viewing device may be an optical device that is incorporated into the shaft of the blunt dissection device. The viewing device can include a distal lens, camera sensor (e.g., CCD or CMOS image sensor) or opening that may be directed to view a surgical space or a surgical site, and a signal transmitter such as an electronic wire or optical fiber extending to the proximal end of the blunt dissection device, or wireless transmitter, to transmit a signal to a location at the device proximal end or to another external location. A signal receiver can be at the proximal end or separate from the device in the form of an image viewer. The viewing device of the blunt dissection device includes a distal end optical viewer that sends an electronic, optical, or wireless signal to the proximal end or to an external viewer (e.g., display), to allow for real-time direct visual communication between the user of the blunt dissection device (e.g., a surgeon) and a surgical site at the distal end of the device.

The viewing device may be incorporated integrally into the blunt dissection device or may be insertable into and removable from an access lumen of the blunt dissection device. According to the former, a flexible optical fiber can extend from the proximal end of the device to the distal end of the device at a location along the shaft, e.g., by being embedded or molded into or against a sidewall of the shaft of the blunt dissection device. The optical fiber can be connected to a lens at the distal end and a viewing device at a proximal end to allows viewing of a space at the distal end of the shaft, e.g., at a surgical site at which an implant will be placed or attached to tissue. Alternately, a viewing device can be in the form of an optical catheter insert (see below) which can include an endoscope that includes a viewing device.

With direct viewing methods based on an optical device feature incorporated into or useful with the described blunt dissection device, a surgeon is able to view internal tissue of a surgical site, preferably an entire range of tissues of a surgical space expanded by insufflation. For performing a surgical procedure of transvaginally placing a supportive implant at a location of a female pelvic region, advantages include the ability to view a wide range of tissues including anterior and posterior tissue locations, to precisely identify relevant tissues. A surgeon is enabled to identify and distinguish specific fascia or muscle tissues at an obturator foramen, e.g., relative to adjacent tissue. The surgeon can likewise identify and distinguish specific apical, posterior, or anterior vaginal tissue, as well as tissues of a sacrospinous ligament, adjacent muscle, and tissue of a region of a sacrum. With the improved ability to view these tissues and distinguish between them, especially in a surgical space opened and expanded by insufflation, the level of control of placement of the implant, sutures, or soft tissue anchors is greatly improved. Also advantageously, the surgeon is better able to avoid accidental contact with other tissues, nerves, or vessels.

The blunt dissection device can be used or provided in combination with one or more separate pieces that are adapted to fit within an access lumen of a blunt dissection device shaft, e.g., to be advance from a proximal device end to the distal device end, and to extend into a surgical space or to a surgical site to provide a desired surgical function. As an example, a blunt dissection device may be used in combination with an endoscope, the endoscope including one or more functionality such as a viewing functionality, a shaft having a length-wise opening adapted to pass surgical items, or a steerable distal end. Other types of separated pieces used or provided in combination with the blunt dissection device can be in the form of a “catheter insert,” which refers to an elongate flexible catheter-sized device that can be passed through the access lumen, placing a distal end of the catheter insert at a surgical site and a proximal end of the catheter insert at a proximal end of the access lumen. The catheter insert can be, for example, adapted from a standard, flexible, catheter having a size that fits into an access lumen of a blunt dissection device. The catheter insert can be of any size desirable for use in a particular procedure, or for use with a particular blunt dissection device and access lumen, with an example of an outer dimension (diameter) being in a range from about 8 to about 18 FR. The catheter insert can be one that provides a desired surgical functionality such as a viewing functionality, an irrigation functionality (i.e., delivery of an irrigation fluid), or a suction functionality (i.e., delivery of reduced pressure, suction, or vacuum to a surgical site).

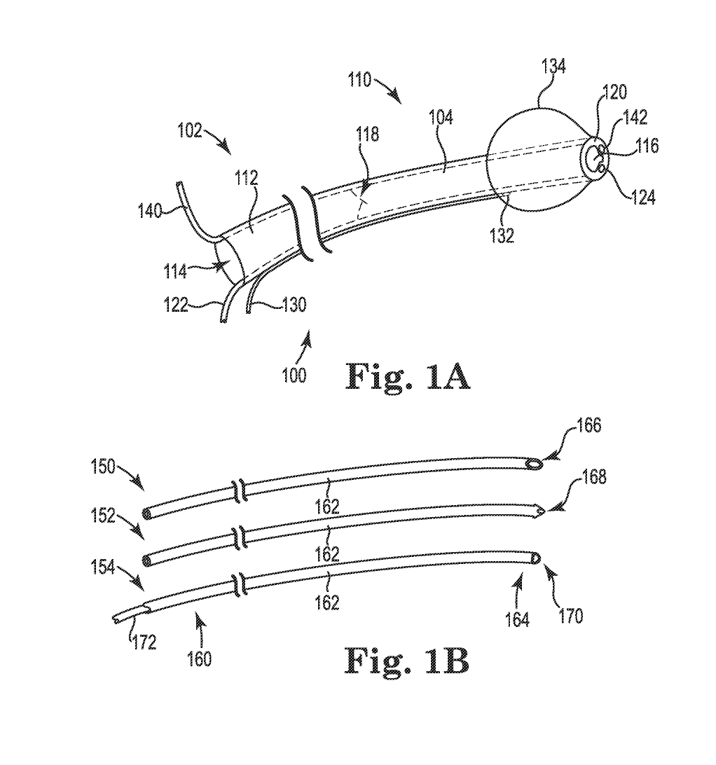

Referring to FIG. 1A, illustrated is blunt dissection device 100, which includes proximal end 102, distal end 110, and flexible shaft 104 extending therebetween. Access lumen 112 is an internal opening or channel extending from the proximal end to the distal end, opening in a distal direction from shaft terminus 120. Access lumen 112 opens at proximal access lumen opening 114 at proximal end 102 and at distal access lumen opening 116 at distal end 110. At a location along the length of access lumen 112 is valve 118, which can maintain a pressure differential between the distal side of the valve 118 and the proximal side of valve 118, the pressure differential being at least a pressure used as an insufflation pressure.

Also extending along the length of shaft 104 between proximal end 102 and distal end 110 is insufflation lumen 122, opening at insufflation lumen opening 124 at shaft terminus 120. As illustrated, insufflation lumen 122 is located within a sidewall of shaft 104. In use, an insufflation fluid (e.g., carbon dioxide) can be passed under pressure through insufflation lumen 122 from proximal end 102 to insufflation lumen opening 124, located at a surgical space, to expand and pressurize the surgical space.

Inflation lumen 130 extends along the length of shaft 104 between proximal end 102 and distal end 110. Inflation lumen 130 is located within a sidewall of shaft 104. Inflation lumen 130 opens at inflation lumen opening 132, which is located at an interior of balloon (expandable surface) 134. In use, an inflation fluid (e.g., air, another gas, or a liquid) can be passed through inflation lumen 130 from proximal end 102 to the interior of balloon 134, under pressure, to expand balloon 134. As desired, the inflation fluid may be released and removed to retract balloon 134.

Still referring to FIG. 1A, device 100 includes an optical viewing device that includes optical fiber 140 extending along the length of shaft 104 between proximal end 102 and distal end 110. Lens 142 at terminus 120 connects to optical fiber 140, to allow optical viewing of a surgical site. As illustrated, the optical viewing device of device 100 is incorporated into a sidewall of shaft 104. In alternate embodiments the viewing device may be separate from shaft 104, e.g., may be inserted through access lumen 104. Also, while the optical viewing device of device 100 is described as including a fiber optic element, alternate viewing devices may include electronic features, a wireless transmitter, or other known viewing device mechanisms useful for collecting an optical signal at distal end 110 and transmitting the signal (optically, electronically, or wirelessly) to a proximal end of device 100 or to an external location in a manner that the signal can be viewed by a user of device 100 to view the surgical site.

FIG. 1B illustrates three different examples of catheter inserts. Each of these catheter inserts 150, 152, and 154, includes a proximal end 160, a distal end 164, and a flexible shaft 162. The outer diameter of the catheter insert is sized to allow the insert to pass through a length of access lumen 112 to pass distal end 164 through lumen 112 and extend distal end 164 through distal access lumen opening 116. Catheter insert 150 includes an open lumen extending along shaft 162 and an opening 166 at distal end 164; insert 150 can be attached to a source of reduced pressure or vacuum to allow for suction at opening 166, which may be passed through access lumen 112 and located at a surgical site. Catheter insert 152 includes an open lumen extending along shaft 162 and an opening in the form of nozzle 168 at distal end 164; insert 150 can be attached to a source of irrigation fluid (e.g., saline) to allow the irrigation fluid to be delivered to a surgical site by passing nozzle 168 through access lumen 112. Catheter insert 154 includes optical fiber 172 extending along shaft 162 and lens 170 at distal end 164; insert 154 can be attached to a proximal end viewing device to allow viewing of a surgical site through lens 170, by passing lens 170 through access lumen 112 and locating lens 170 at the surgical site. Any of these exemplary catheter inserts may have a steerable distal end.

In certain device and method embodiments, the blunt dissection device may be used to place an implant at a location of a pelvic region, to treat a pelvic condition. Any of various implants, assemblies, and systems can be placed using a device or method as described herein, in the treatment of a pelvic condition such as fecal or urinary incontinence including stress urinary incontinence (SUI), vaginal prolapse, a condition of the pelvic floor, erectile dysfunction, and the like. An implant or a component of an implant can be surgically placed in a male or a female patient to treat a condition such as urge incontinence; stress urinary incontinence; mixed incontinence; overflow incontinence; functional incontinence; fecal incontinence; prolapse (e.g. vaginal or uterine); enterocele (e.g. of the uterus); rectocele; cystocele; anatomic hypermobility; erectile dysfunction, or a condition of the pelvic floor caused by weakness or trauma of pelvic floor muscles such as the levator (“levator ani”) or coccygeus muscle (collectively the pelvic floor); other conditions caused by muscle and ligament weakness; and combinations of these.

Examples of useful implants for supporting pelvic tissue (e.g., vaginal tissue) can include a tissue support portion that can be used to support pelvic tissue such as the urethra (which includes the bladder neck), bladder, rectum, vaginal tissue (Level 1, Level 2, Level 3, or combinations of these), pelvic floor tissue (levator muscle), etc. During use, the tissue support portion is typically placed in contact with and attached to tissue to be supported, such as by attachment using one or more sutures. The implant can additionally include one or more extension portions attached to the tissue support portion.

Examples of implants and useful for treating pelvic conditions include those commercially available from the American Medical Systems under the trade names MiniArc™, Elevate™, and Y-Mesh™, and those described in U.S. Pat. Nos. and Patent Application documents 2010/0256422; 6,592,515; 8,388,514; and 8,597,173, the entireties of these documents being incorporated herein by reference.

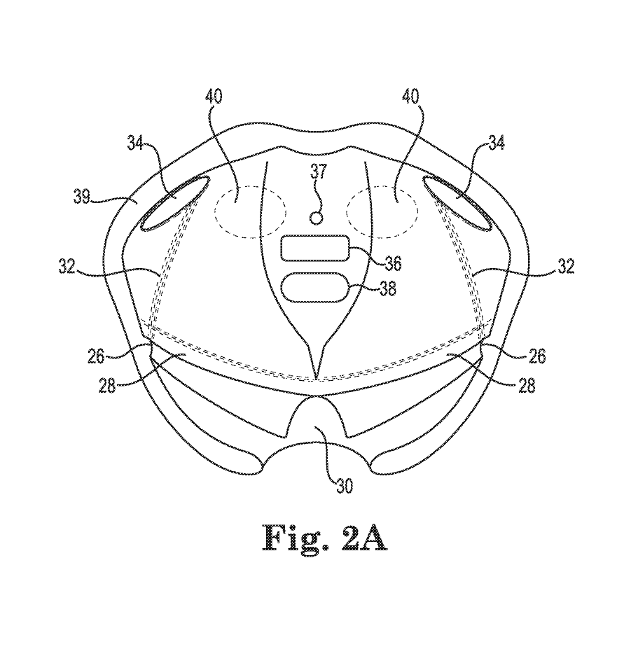

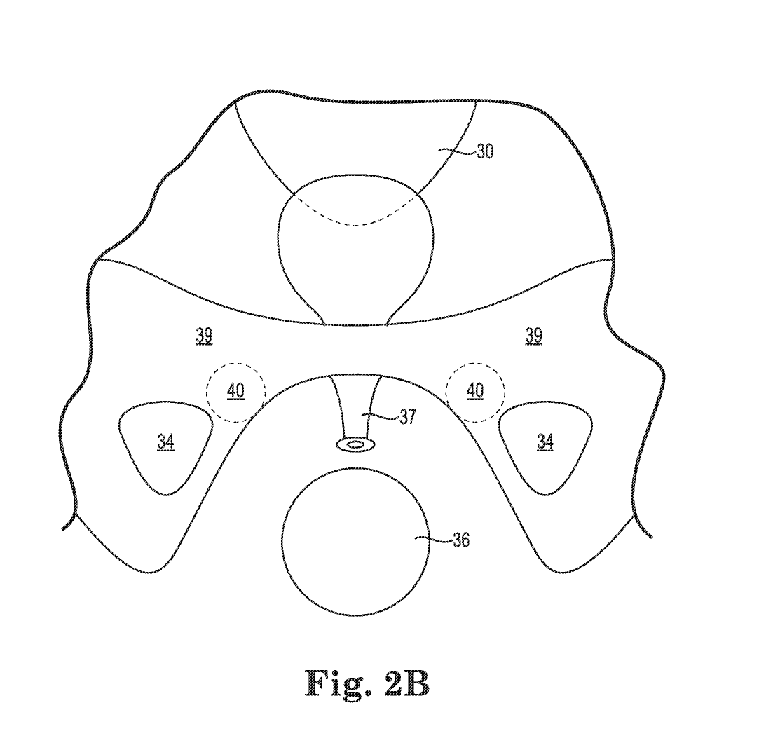

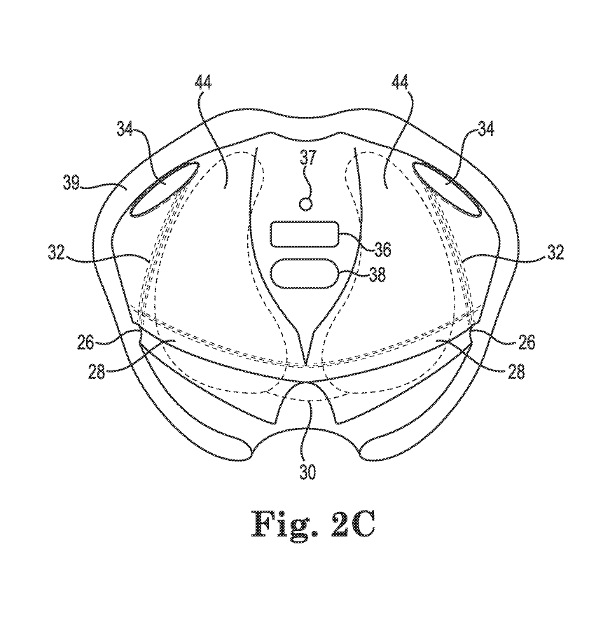

The inventive devices can be useful in methods for blunt dissection of tissue, including but not limited to treatments of conditions of urinary incontinence or vaginal prolapse at a female pelvic region, e.g., wherein the procedure is performed transvaginally. With reference to FIGS. 2A, 2B, and 2C, illustrated are various views of a female pelvic region. These figures show anatomy relevant to methods and devices of embodiments of described methods for treating a female pelvic condition. In particular, FIG. 2A illustrates a superior view of tissue at different levels of a female pelvic region including ischial spine 26, coccyx 30, arcus tendineus 32, obturator foramen 34, vagina 36, urethra 37, rectum 38, and pelvic bone 39. FIG. 2B illustrates these tissues from an anterior perspective of the patient, also showing left and right dissection sites 40 located behind (posterior to) an ischial surface of a subpubic arch.

According to methods of treating a pelvic region of a female patient, a blunt dissection devices as described herein can be useful to perform a transvaginal step of blunt dissection at a dissection site in a location of the pelvic region. The surgical site at the location of the pelvic region can then be insufflated and optionally and preferably can be viewed using a viewing device integral to or combinable with the blunt dissection device, during a surgical procedure such as placement of a supportive implant.

In a female pelvic procedure, effective locations for blunt dissection using the expandable surface include locations in the retroischial space, to dissect and expand tissue within the potential interfacial planes. A preferred location for blunt dissection is the “interfacial plane” (a.k.a. “submucosal plane”), meaning a plane defined to include a location directly along the vaginal wall, between tissue of an anterior vaginal tissue and the bladder and bladder neck, and that extends on both sides of a patient in anterior direction to the pubic bone; laterally to the opposed obturator foramen, arcus tendineus and ischial spine; and posteriorly to the sacrospinous ligament and sacral region, including the coccyx bone. This plane includes an interface between tissues, the interface being generally avascular, and, advantageously, separable by blunt dissection without the need for cutting tissue using a sharp cutting instrument. Blunt dissection of the avascular interfacial plane, without cutting, offers the advantage of a relatively bloodless and efficient blunt dissection step, and after blunt dissection can be insufflated to produce a large surgical space that provides good access to many different tissues of the female pelvic region, such as one or more of the pubic bone, pelvic bone, ischial spine, sacrospinous ligament, white line (arcus tendineus), pelvic surface of the obturator foramen covered with obturator fascia, bladder or bladder neck, urethra and adjacent fascia, levator muscle, perineal body, uterosacral ligament, sacrum, or coccyx bone, muscles and ligaments adjacent to any of these structures, among others.

Generally, methods of using the described blunt dissection device for treating a pelvic region of a female patient can begin with an incision made in vaginal tissue, such as anterior vaginal tissue. From that incision, vaginal wall flaps can be developed by carrying out the dissection in the submucosal plane between the vagina and tissues of the bladder, bladder neck, and urethra. This dissection is further carried out cranially up to vaginal apex. A distal end of the blunt dissection device is passed through the patient's vaginal opening, is advanced through the vaginal canal, through the vaginal incision, and is placed at a desired location for a blunt dissection, i.e., a dissection site, typically on a left or right side of the patient.

Optionally and preferably, a surgeon can perform blunt dissection first on one side of the patient (the left or right side), followed by a separately-performed blunt dissection of tissue on the other side, as opposed to a blunt dissection step at a single medial location. The blunt dissection step may preferably be performed by inflating and deflating the expandable surface two or more times per side of the patient, progressively advancing the expandable surface into the dissected space with expansion and retraction of the expandable surface, optionally with assistance using the surgeon's fingers. For example, once the expandable surface has initially passed through the vaginal incision to become located in the pelvic region, e.g., on one side of the patient, the surgeon may expand the expandable surface to create an initial space. The surgeon may advance the expanded surface manually from the initial space, optionally in the expanded state or optionally by deflating (retracting) the expandable surface, advancing the distal end of the blunt dissection device to an advanced location in the pelvic region of the patient with the expandable surface in a retracted state, and then re-expanding the expandable surface at the more advanced location to progressively open additional space in the patient.

As a specific example of blunt dissection of a female pelvic region, a small space can first be created behind an ischial surface of a subpubic arch on a first side of a female patient. These spaces are designated as numbers 40 at FIGS. 2A and 2B. The expandable surface of the distal end of the blunt dissection device is placed in or about this space (40) on one side of the patient. This space 40 is then opened by expanding the expandable surface in a gradually increasing manner. After attaining a certain desired degree of expansion, the expandable surface is left expanded for some time (such as a time in a range from about 2 to about 3 minutes). Bleeding, if any, can be observed as drainage through an optional access lumen of the blunt dissection device, by visualization using a viewing device of the blunt dissection device, or both. The expandable surface is then deflated. The distal end of the blunt dissection device is advanced to a deeper location in the pelvic region of the patient. The expandable surface is then re-expanded. This sequence of steps may be repeated multiple times to achieve a desired blunt dissection effect, on both the right and the left sides of the patient.

Optionally, a surgical space created as described by blunt dissection using the blunt dissection device can be expanded by causing an insufflation gas to flow into the dissected space, under pressure. This also allows for deflation (retraction) of the balloon (expandable surface). Methods of insufflation, insufflation gases, and techniques and equipment to perform insufflation steps are known and will be understood to be adaptable to a transvaginal or other type of blunt dissection procedure described herein. With respect to a transvaginal method of blunt dissection followed by insufflation, the described blunt dissection device can be placed transvaginally through the external vaginal opening, vaginal canal, and a vaginal incision, and the internal space of the pelvic region that is dissected can be sufficiently air-tight to allow for insufflation as described.

FIG. 2C shows an example of a well-extended dissected space as described and illustrated at FIG. 2A, after blunt dissection and insufflation. Spaces 44 on the left and right sides of the patient allow access to tissue that includes the vaginal tissue 36, both obturator foramen 34, each white line (arcus tendineus) 32, both ischial spines 26, sacrospinous ligament 28, and coccyx 30. Dissection spaces as defined by spaces 44 of FIG. 2C are exemplary. As illustrated, spaces 44 may be the largest boundary of a surgical space created by transvaginal blunt dissection of a female pelvic region. A surgical space that is not as large as that shown at FIG. 2C is also useful, e.g., a surgical space that is smaller than a space 44 of FIG. 2C but larger than a space 40 of FIG. 2A.

After the step of blunt dissection of the retroischial space, e.g., as shown at FIG. 2C, a surgeon is able to perform subsequent steps of a surgical treatment method, such as identifying and accessing tissue adjacent to the dissected and expanded space for placement of a supportive implant. For various treatments of anterior female pelvic conditions, a surgeon may wish to identify and access one or more of the ischial spine, sacrospinous ligament, and the pelvic surface of the obturator foramen covered with obturator fascia. These anatomical locations may be identified and used for placing and attaching an element (e.g., suture or soft tissue anchor) of a supportive sling implant.

Desirably, the surgical procedure may be performed using an embodiment of blunt dissection device that includes an insufflation lumen, an access lumen, and a distal end viewing device (e.g., integrated within the device shaft). With this type of blunt dissection tool, and features thereof, the dissected space can be insufflated to create a dissected and expanded space for performing a surgical procedure such as placement of a supportive sling implant. During placement of the implant, the surgeon may view the dissected space and any tissue therein using the distal end viewing feature. Additionally, the surgeon may access the surgical site and present surgical items such as an implant or pieces of an implant through the access lumen. Alternately, subsequent to the blunt dissection step and insufflation step, a flexible endoscope or other surgical tool may be introduced through the surgical incision to place a distal end of the endoscope or other tool at the dissected surgical site to visualize the dissected and insufflated space and assist in performing steps for placing the supportive implant. After the implant is successfully placed, the balloon is finally deflated and the blunt dissection device can be withdrawn.

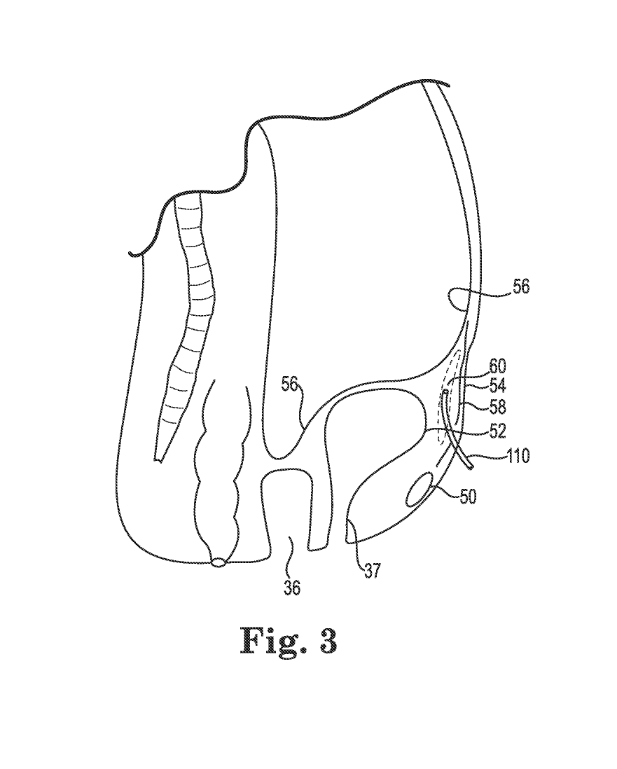

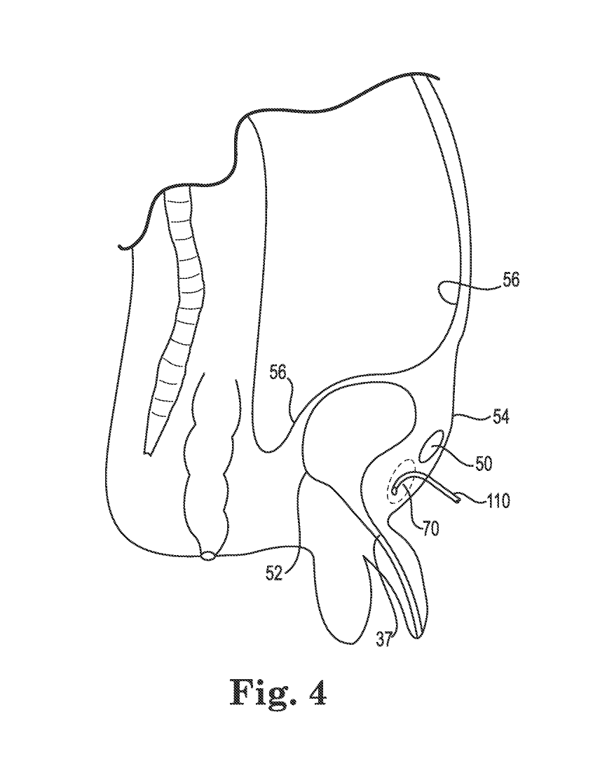

According to other methods of the invention, using the described blunt dissection device, extraperitoneal procedures (i.e., procedures that do not breach, cut, or traverse the peritoneum) can be performed at an abdominal location in a male or female patient. With reference to FIG. 3, illustrated is anatomy of a pelvic region viewed as a sagittal plane at a medial location of a female patient. FIG. 3 shows anatomy relevant to methods and devices of embodiments of described methods for treating a male or female condition of an abdomen, such as a hernia in a male or female. In particular, FIG. 3 illustrates a medial plane of tissue a female pelvic region including pubic bone (or pubic symphysis) 50, bladder 52, epidermis 54, peritoneum 56, fat and muscle layer 58 of an abdominal wall, vagina 36, and urethra 37.

Also shown at FIG. 3 is a distal end 110 of a blunt dissection device 100, as described. FIG. 3 illustrates distal end 110, including an expandable surface (not shown), passing through an external incision at epidermis 54, through abdominal fat and muscle layer 58, and to a general dissection location 60 between peritoneum 56 and abdominal layer 56.

Once device 100 is placed as shown at FIG. 3, a surgeon can perform blunt dissection separately at a left side, at a right side of the patient, or at a medial location of the patient, as desired for a given condition and treatment. Blunt dissection may be performed by inflating and deflating the expandable surface two or more times per desired dissection site and progressively advancing the expandable surface into the dissected space, optionally with assistance using the surgeon's fingers. Once the expandable surface has initially passed through the external incision to become located at a desired location between peritoneum 58 and abdominal layer 58, the surgeon may expand the expandable surface to create an initial space. The surgeon may advance the expanded surface manually, optionally in the expanded state or optionally by deflating the expandable surface, advancing the distal end of the blunt dissection device to an advanced location in the pelvic region of the patient, then re-expanding the expandable surface at the more advanced location to progressively open space in the patient. This sequence of steps may be repeated multiple times to achieve a desired blunt dissection effect, and on both the right and left sides of the patient. After the blunt dissection step, the blunt dissection device may optionally be used to insatiate the dissected space.

After the step of blunt dissection space 60 and optional insufflation, a surgeon will be able to perform subsequent steps of a desired surgical treatment, such as accessing tissue adjacent to the dissected (and optionally expanded) space. For repair of a hernia, a surgeon may wish to identify, access, and repair muscle of the abdominal wall (layer 54). Desirably, the surgical procedure may be performed using an embodiment of blunt dissection device that includes an insufflation lumen, an access lumen, and a distal end viewing feature. With this blunt dissection tool, the dissected space 60 can be insufflated to create a dissected and expanded space for performing a procedure (e.g., hernia repair). During placement of surgical items such as sutures, tools, or mesh repair implants, the surgeon may view the dissected and insufflated space 60 and any tissue therein using the distal end viewing feature. Additionally, the surgeon may access the surgical site and present surgical items such as a suture, an implant, or pieces of an implant, through the access lumen. According to alternate embodiments, subsequent to the blunt dissection step and insufflation step, a flexible endoscope or other surgical tool may be introduced through the surgical incision to place a distal end of the endoscope or other tool at the dissected surgical site to visualize the dissected and insufflated space and assist in performing steps for placing the supportive implant. After the desired procedure is completed the balloon can be deflated and the blunt dissection device can be withdrawn.

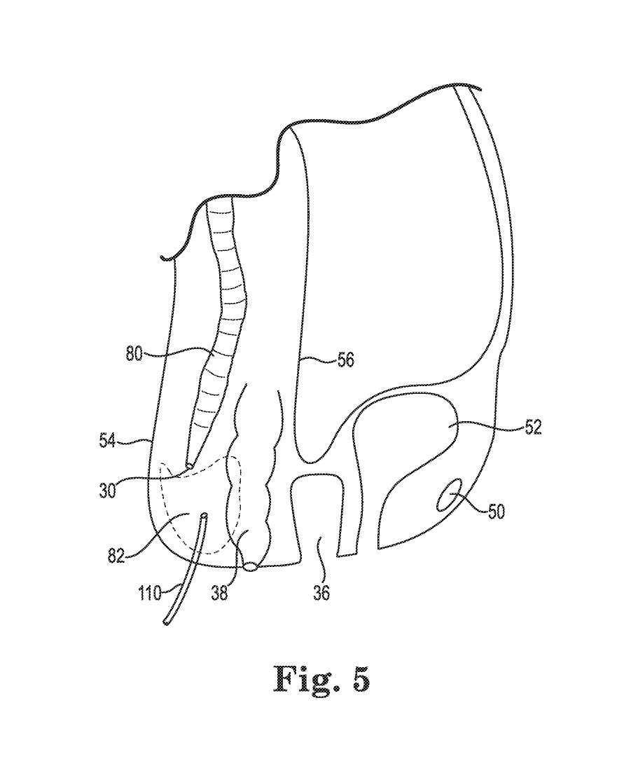

In another method embodiment, as shown at FIG. 4 a blunt dissection device 100 may be used to prepare dissected space 70, located generally in a male abdominal space anterior to bladder 52, superior to urethra 37, and posterior or inferior to pubic bone 50. Space 70 can be prepared for placement of a reservoir of an implantable penile pump, or alternately an artificial urinary sphincter. Blunt dissection of space 70 can be performed by steps as described herein, of advancing distal end 110 through an external incision in skin 54 and expanding the expandable surface in conjunction with advancing the distal end to create and expand space 70. Once space 70 is created a surgeon may perform steps of accessing tissue adjacent to space 70, optionally with distal end 110 remaining in the patient or with removal of distal end 110.

Once device 100 is placed as shown at FIG. 4, a surgeon can perform blunt dissection separately of a left side, a right side of the patient, or at a medial location of the patient, as desired for a given condition and treatment. Blunt dissection may be performed by inflating and deflating the expandable surface two or more times per side of the patient, and progressively advancing the expandable surface into the dissected space, optionally with assistance using the surgeon's fingers. Once the expandable surface has initially passed through the external incision to become located at a desired location of space 82, the surgeon may expand the expandable surface to create an initial space. The surgeon may advance the expanded surface manually, optionally in the expanded state or optionally by deflating the expandable surface, advancing the distal end of the blunt dissection device to an advanced location in the pelvic region of the patient, then re-expanding the expandable surface at the more advanced location to progressively open space in the patient. This sequence of steps may be repeated multiple times to achieve a desired blunt dissection effect, and on both the right and left sides of the patient.

After the step of blunt dissection of space 70, a surgeon will be able to perform subsequent steps of a desired surgical treatment, such as accessing tissue adjacent to the dissected and expanded (e.g., retropubic) space 70. Desirably, the surgical procedure may be performed using an embodiment of blunt dissection device that includes an insufflation lumen, an access lumen, and a distal end viewing feature. With these features of a blunt dissection tool, dissected space 70 can be insufflated to create a dissected and expanded space 70 for performing the surgical procedure. During placement of relevant items such as a suture, surgical tool, implant component (e.g., reservoir or a penile prosthesis, or artificial urinary sphincter), the surgeon may view the dissected and insufflated space 70 and any tissue therein using the distal end viewing feature. Additionally, the surgeon may access the surgical site and present surgical items such as a suture, an implant, or pieces of an implant through the access lumen. According to alternate embodiments, subsequent to the blunt dissection step and insufflation step, a flexible endoscope or other surgical tool may be introduced through the surgical incision to place a distal end of the endoscope or other tool at the dissected surgical site to visualize the dissected and insufflated space and assist in performing steps for placing the supportive implant. After the desired procedure is completed the expandable surface (e.g., balloon) can be deflated and the blunt dissection device can be withdrawn.

FIG. 5 shows alternate methods the invention, for using the described blunt dissection device in a posterior, extraperitoneal procedure to access tissue on a posterior side of a pelvic region such as a sacrum, rectum, rectal sphincter, or adjacent muscle such as levator muscle, in a male or female patient. With reference to FIG. 5, illustrated is anatomy of a pelvic region viewed as a sagittal (medial) plane. FIG. 5 shows anatomy relevant to methods for treating a condition of an extraperitoneal posterior pelvic region in a male or female patient, such as a condition of the rectum, anal sphincter, levator or adjacent muscle, or adjacent muscle tissue. In particular, FIG. 5 illustrates a sagittal plane of tissue a female pelvic region including pubic bone (or pubic symphysis) 50, bladder 52, epidermis 54, peritoneum 56, urethra 37, rectum 38, and spine 80. Also shown at FIG. 5 is a distal end 110 of a blunt dissection device 100, as described. FIG. 5 illustrates distal end 110, including an expandable surface (not shown), passing through an external incision at epidermis 54 and to a general location 82 adjacent to rectum 38 and at an extraperitoneal position between peritoneum 56 and epidermis 54.

Once device 100 is placed as shown at FIG. 5, a surgeon can perform blunt dissection separately of a left side, a right side of the patient, or at a medial location of the patient, as desired for a given condition and treatment. Blunt dissection may be performed by inflating and deflating the expandable surface two or more times per side of the patient, and progressively advancing the expandable surface into the dissected space, optionally with assistance using the surgeon's fingers. Once the expandable surface has initially passed through the external incision to become located at a desired location of space 82, the surgeon may expand the expandable surface to create an initial space. The surgeon may advance the expanded surface manually, optionally in the expanded state or optionally by deflating the expandable surface, advancing the distal end of the blunt dissection device to an advanced location in the pelvic region of the patient, then re-expanding the expandable surface at the more advanced location to progressively open space in the patient. This sequence of steps may be repeated multiple times to achieve a desired blunt dissection effect, and on both the right and left sides of the patient.

After the step of blunt dissection of space 82, a surgeon will be able to perform subsequent steps of a desired surgical treatment, such as accessing tissue adjacent to the dissected and expanded space 82, e.g., rectum 38, spine 80, or coccyx 30, adjacent muscle such as levator ani (not specifically shown), or an anal sphincter (not shown). For a surgical procedure at space 82, a surgeon may wish to identify, access, and repair any one or more of these tissues. Desirably, the surgical procedure may be performed using an embodiment of blunt dissection device that includes an insufflation lumen, an access lumen, and a distal end viewing feature. With these features of a blunt dissection tool, dissected space 82 can be insufflated to create a dissected and expanded space for performing the surgical procedure. During placement of relevant items such as a suture, surgical tool, or mesh repair implant, the surgeon may view the dissected and insufflated space 82 and any tissue therein using the distal end viewing feature. Additionally, the surgeon may access the surgical site and present surgical items such as a suture, an implant, or pieces of an implant through the access lumen. According to alternate embodiments, subsequent to the blunt dissection step and insufflation step, a flexible endoscope or other surgical tool may be introduced through the surgical incision to place a distal end of the endoscope or other tool at the dissected surgical site to visualize the dissected and insufflated space and assist in performing steps for placing the supportive implant. After the desired procedure is completed the balloon can be deflated and the blunt dissection device can be withdrawn.

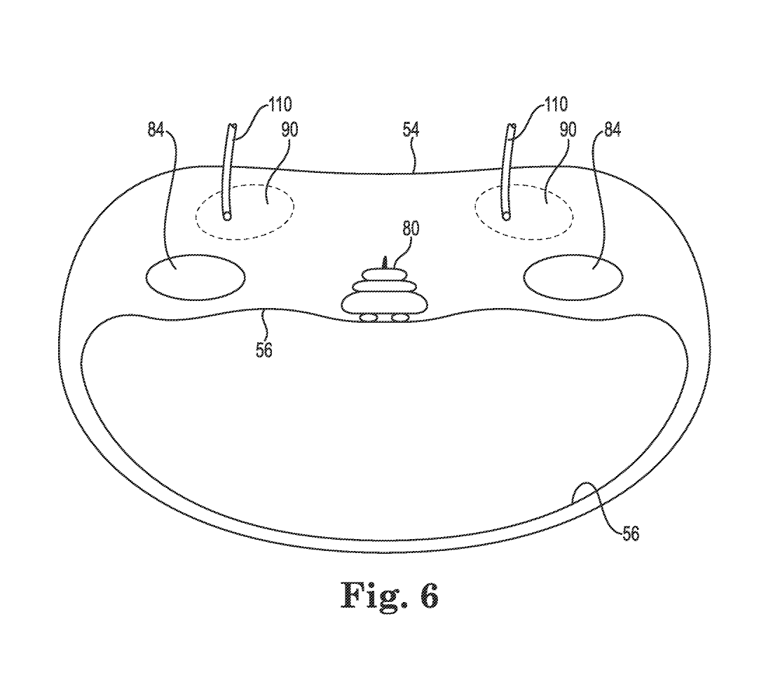

FIG. 6 shows yet another method the invention, this one for using the described blunt dissection device in an extraperitoneal procedure to access a kidney 84. With reference to FIG. 6, illustrated is anatomy of a transverse plane at a level of a kidney. FIG. 6 shows anatomy relevant to methods for treating a condition of a kidney. In particular, FIG. 6 illustrates a transverse plane of tissue a male or female patient, including kidneys 84, spine (vertebra) 80, epidermis 54, and peritoneum 56. Also shown at FIG. 6 is a distal end 110 of a blunt dissection device 100, as described. FIG. 6 illustrates distal end 110, including an expandable surface (not shown), passing through an external incision at epidermis 54 and to a general location 90 adjacent to a kidney 84 at an extraperitoneal position between peritoneum 56 and epidermis 54.

Once device 100 is placed as shown at FIG. 6, a surgeon can perform blunt dissection separately on a left side or a right side of the patient in a region of a left or a right kidney 84. Blunt dissection may be performed by inflating and deflating the expandable surface two or more times per side of the patient, and progressively advancing or moving (left, right, up, or down) the expandable surface into the dissected space. Once the expandable surface has initially passed through the external incision to become located at a desired location of space 90, the surgeon may expand the expandable surface to create an initial space. The surgeon may advance or move the expanded surface manually, optionally in the expanded state or optionally by deflating the expandable surface, advancing the distal end of the blunt dissection device to an advanced location in the pelvic region of the patient, then re-expanding the expandable surface at the more advanced location to progressively open space in the patient. This sequence of steps may be repeated multiple times to achieve a desired blunt dissection effect, and a right or a left side of the patient.

After the step of blunt dissection of space 90, a surgeon will be able to perform additional steps of a desired surgical treatment, such as accessing tissue adjacent to the dissected and expanded space 90, e.g., kidney 84 or adjacent tissue. For a surgical procedure at space 90, a surgeon may wish to identify and access tissue of kidney 84 or tissue adjacent to a kidney 84. Desirably, the surgical procedure may be performed using an embodiment of blunt dissection device that includes an insufflation lumen, an access lumen, and a distal end viewing feature. With such a blunt dissection tool, the dissected space 90 can be insufflated to create a dissected and expanded space for performing the surgical procedure. During placement of relevant items such as a suture, surgical tool, or mesh repair implant, the surgeon may view the dissected and insufflated space 90 and any tissue therein using the distal end viewing feature. Additionally, the surgeon may access the surgical site and present surgical items such as sutures, an implant, pieces of an implant through the access lumen. According to alternate embodiments, subsequent to the blunt dissection step and insufflation step, a flexible endoscope or other surgical tool may be introduced through the surgical incision to place a distal end of the endoscope or other tool at the dissected surgical site to visualize the dissected and insufflated space and assist in performing steps for placing the supportive implant. After the desired procedure is completed the balloon can be deflated and the blunt dissection device can be withdrawn.

In still alternate embodiments of method for using a blunt dissection device as described, the device may be useful for blunt dissection of any tissue or any two adjacent tissues. The device may be useful in a method by which a surgeon creates a space in a tissue, especially a space between separable tissue (including organs) items to gain access to one or both of the different tissues. Examples of two adjacent tissues that may be separated by use of a blunt dissection device as described include any two tissues that are known to be capable of being dissected by blunt dissection techniques such as by digital dissection, balloon dissection, insufflation, etc. Some non-limiting examples including: adjacent bone and muscle tissues, peritoneum tissue and an adjacent muscle or fatty tissue, a muscle and an organ, and muscle and fascia.

1. A blunt dissection device comprising:
a proximal device end;
a distal device end;
a flexible shaft extending along a length between the proximal device end and the distal device end;
an expandable surface extending along a length of the shaft at the distal device end, expandable from the shaft;
a lumen extending along a length of the shaft in operative communication with the expandable surface; and
an insufflation lumen extending along a length of the shaft between the proximal end and the distal end.

2. A blunt dissection device comprising:
a proximal device end;
a distal device end;
a flexible shaft extending along a length between the proximal device end and the distal device end;
an expandable surface extending along a length of the shaft at the distal device end, expandable from the shaft;
a lumen extending along a length of the shaft in operative communication with the expandable surface; and
an access lumen within the shaft extending from the proximal device end to an access opening at a distal end of the shaft.

3. A blunt dissection device as recited at claim 1 comprising a source of pressurized insufflation gas in communication with the insufflation lumen.

4. A blunt dissection device as recited at claim 1 comprising an access lumen within the shaft extending from the proximal device end to an distal access lumen opening at a distal end of the shaft, wherein the distal access opening is on a distal side of the expandable surface.

5. A blunt dissection device as recited at claim 1 comprising a viewing device capable of viewing a surgical site at the distal device end and transmitting a signal of the site to a display device.

6. A blunt dissection device as recited at claim 1 wherein the expandable surface is an inflatable balloon and the lumen is an inflation lumen that fluidly connects a sealed interior of the balloon to the proximal end.

7. A blunt dissection device as recited at claim 1 wherein the expandable surface can be expanded to a volume in a range from about 60 to about 90 cubic centimeters.

8. A blunt dissection device as recited at claim 2 wherein the shaft is flexible and has a substantially uniform outer diameter in a range from about 4 to about 10 millimeters, and a the access lumen has a substantially uniform diameter in a range from about 4 about 8 millimeters.

9. A blunt dissection device as recited at claim 2 in combination with a catheter insert that is passable through the access lumen, the catheter insert being selected from the group consisting of an irrigation insert capable of providing irrigation fluid at a distal end of the catheter insert, a viewing device, and a suction insert capable of providing suction at a distal end of the catheter insert.

10. A blunt dissection device as recited at claim 2 comprising a distal access lumen opening at a terminus of the shaft.

11. A blunt dissection device as recited at claim 10 wherein the opening is oriented transversely to a longitudinal axis of the shaft.

12. A blunt dissection device as recited at claim 1 wherein the expandable surface comprises a distal end and a proximal end, the shaft comprises a terminus, and the distal end of the expandable surface is located a distance of not more than 5 millimeters from the terminus.

13. A method of blunt dissection, the method comprising:
providing a blunt dissection device as recited at claim 1,
making an incision in a patient in a patient,
passing the device distal end through the incision,
advancing the device distal end to place the expandable surface at a dissection site, and
expanding the expandable surface to cause blunt dissection of tissue at the dissection site.

14. A method as recited at claim 13 wherein the incision is an anterior vaginal incision.

15. A method as recited at claim 13 wherein the dissection site is at tissue in a submucosal plane located between anterior vaginal tissue and a bladder or urethra and the method comprises expanding the expandable surface to produce a space between the anterior vaginal tissue and the bladder or urethra.

16. A method as recited at claim 15 comprising performing blunt dissection of the submucosal plane on a right side of the patient, and separately performing blunt dissection of the submucosal plane on a left side of the patient.

17. A method as recited at claim 13 wherein the device comprises a viewing device, and the method comprises using the viewing device to view the dissection site.

18. A method as recited at claim 13 wherein the blunt dissection device comprises an insufflation lumen that fluidly connects an insufflation opening at the device distal end to a source of pressurized insufflation gas at the proximal end, the method comprising passing insufflation gas through the insufflation lumen, under pressure, into the dissection site.

19. A method as recited at claim 13 wherein the dissection site is located posterior to an ischial surface of a subpubic arch.

20. A method as recited at claim 13 comprising treating a condition selected from anterior vaginal prolapse and posterior vaginal prolapse.
